(19) United States
(12) Patent Application Publication
(10) Pub. No.: US 2022/0220123 A1
(43) Pub. Date: Jul. 14, 2022

(54) AMORPHOUS AND CRYSTALLINE FORMS OF RELUGOLIX

(71) Applicant: DR. REDDY'S LABORATORIES LIMITED, Hyderabad (IN)

(72) Inventors: Jayprakash Amarpal YADAV, Taluka: Vaso (IN); Srinivas ORUGANTI, Hyderabad (IN); Saikat SEN, Kolkata (IN); Satyanarayana THIRUNAHARI, Hyderabad (IN); Satish Chowdary NEKKANTI, Hyderabad (IN); Shanmukha Prasad GOPI, Vishakapatnam (IN)

(21) Appl. No.: 17/610,052

(22) PCT Filed: May 15, 2020
(86) PCT No.: PCT/IB2020/054598

§ 371 Date: Nov. 9, 2021

(30) Foreign Application Priority Data
May 15, 2019 (IN) 201911019300
May 30, 2019 (IN) 201941021467
Jun. 14, 2019 (IN) 201941023637
Jul. 18, 2019 (IN) 201941028925
Sep. 3, 2019 (IN) 201941035392
Oct. 16, 2019 (IN) 201941041885
Dec. 20, 2019 (IN) 201941053151

(51) Int. Cl. C07D 495/04

(52) U.S. Cl. CPC C07D 495/04; C07B 2200/13

(57) ABSTRACT

The present application relates to crystalline forms R1-R10 of relugolix and process for preparation thereof. The present application also relates to amorphous form and amorphous solid dispersion of relugolix and process for preparation thereof. Crystalline forms R1 are stable under all storage conditions for three months. Crystalline form R1 have excellent physico-chemical properties and may be used conveniently in a pharmaceutical composition comprising relugolix. The application also relates to a process for purification of relugolix.

### FIELD OF INVENTION

The present application is directed to crystalline forms R1-R10 of relugolix and process for preparation thereof. The present application is also directed to amorphous relugolix and amorphous solid dispersion of relugolix and process for preparation thereof. Moreover, the present application is directed to a process for purification of relugolix.

Relugolix is a gonadotropin-releasing hormone antagonist (GnRH antagonist) which is under development for use in treatment of endometriosis and prostate cancer. Relugolix is chemically known as N-[4-[1-[(2,6-difluorophenyl)methyl]-5-[(dimethylamino]-methyl]-3-(6-methoxy-3-pyridazinyl]-2,4-dioxo-1,2,3,4-tetrahydro-thieno[2,3-d]pyrimidin-6-yl]phenyl]-N′-methoxyurea and has following structural formula:

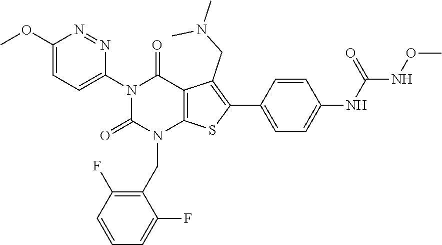

PCT patent application, WO2004067535A1 (hereinafter referred as the WO'535 application) discloses relugolix specifically for the first time.

PCT patent application, WO2014051164A2 (hereinafter referred as the WO'164 application) discloses two crystalline forms of relugolix, characterized by FIGS. 1 and 2. The crystalline form of FIG. 1 has been characterized as tetrahydrofuran solvate, however no characterization is given for FIG. 2.

In general, polymorphism refers to the ability of a substance to exist as two or more crystalline phases that have different spatial arrangements and/or conformations of molecules in their crystal lattices. Thus, “polymorphs” refer to different crystalline forms of the same pure substance in which the molecules have different spatial arrangements of the molecules, atoms, and/or ions forming the crystal. Different polymorphs may have different physical properties such as melting points, solubilities, X-ray diffraction patterns, etc. The variation in solid forms may appreciably influence the pharmaceutical properties, such as bioavailability, handling properties, dissolution rate, and stability, and in turn such properties can significantly influence the processing, shelf life, and commercial acceptance of a polymorphic form. For these reasons, regulatory authorities require drug manufacturing companies to put efforts into identifying all polymorphic forms, e.g., crystalline, amorphous, solvates, solid dispersions with pharmaceutically acceptable carriers, etc., of new drug substances.

The existence and possible numbers of polymorphic forms for a given compound cannot be predicted, and there are no “standard” procedures that can be used to prepare polymorphic forms of a substance. This is well-known in the art, as reported, for example, by A. Goho, “Tricky Business,” Science News, Vol. 166(8), August 2004.

It has been disclosed earlier that the amorphous forms in a number of drugs exhibit different dissolution characteristics and in some cases different bioavailability patterns compared to crystalline forms [Konne T., Chem pharm Bull., 38, 2003(1990)]. Typically, the more crystalline the pharmaceutical agent, the lower is its bioavailability or vice varsa, reducing the degree of crystallinity has a positive effect on bioavailability. Amorphous material generally offers interesting properties such as higher dissolution rate and solubility than crystalline forms, typically resulting in improved bioavailability. An amorphous form of cefuroxime axetil is a good example for exhibiting higher bioavailability than the crystalline form.

Hence, there remains a need for alternate solid forms of relugolix and processes for preparing them.

### SUMMARY OF THE INVENTION

First aspect of the present application relates to amorphous form of relugolix.

Second aspect of the present application relates to process for preparing amorphous form of relugolix comprising
(a) dissolving relugolix in a suitable solvent or mixture thereof,
(b) removing the solvent of step (a) by any suitable technique, and
(c) optionally drying amorphous relugolix.

Third aspect of the present application relates to a pharmaceutical composition comprising amorphous form of relugolix and one or more pharmaceutically acceptable excipient.

Fourth aspect of the present application relates to amorphous solid dispersion of relugolix with one or more pharmaceutically acceptable carrier.

Fifth aspect of the present application relates to process for preparation of amorphous solid dispersion of relugolix with one or more pharmaceutically acceptable carrier comprising
(a) dissolving relugolix and one or more pharmaceutically acceptable carrier in suitable solvent or mixture thereof;
(b) isolating amorphous solid dispersion of relugolix;
(c) optionally drying the amorphous solid dispersion of relugolix.

Sixth aspect of the present application relates to a pharmaceutical composition comprising amorphous solid dispersion of relugolix and one or more pharmaceutically acceptable excipient.

Seventh aspect of the present application relates to crystalline form R1 of relugolix characterized by its powder X-ray diffraction (PXRD) pattern having peaks at 7.076, 9.632, 13.048, 15.795 and 20.872±0.2° 2θ. In embodiments, the present application provides crystalline form R1 of relugolix characterized by its PXRD pattern having additional peaks located at 10.665, 12.424, 18.892, 19.216, 23.544, 26.612 and 26.641±0.2° 2θ.

Eighth aspect of the present application provides crystalline form R1 of relugolix characterized by a PXRD pattern substantially as illustrated in FIG. 8.

Ninth aspect of the present application provides a process for preparing crystalline form R1 of relugolix comprising:

a) providing a solution of relugolix in a suitable solvent or mixtures thereof;
b) stirring the solution for sufficient time; and
c) isolating crystalline form R1 of relugolix.

Tenth aspect of the present application relates to a composition comprising crystalline form R1 of relugolix and one or more pharmaceutically acceptable excipient.

Eleventh aspect of the present application relates to a process for purification of relugolix comprising the steps of:

a) providing a mixture of crude relugolix and a suitable solvent;
b) optionally adding an anti-solvent to the mixture of step a);
c) isolating pure relugolix.

Twelfth aspect of the present application relates to crystalline form R2 of relugolix characterized by its powder X-ray diffraction (PXRD) pattern having peaks at 9.404, 18.032 and 19.059±0.2° 2θ. In embodiments, the present application provides crystalline form R2 of relugolix characterized by its PXRD pattern having additional peaks located at 7.438, 10.133, 12.058, 16.488, 21.022 and 23.062±0.2° 2θ.

Thirteenth aspect of the present application provides crystalline form R2 of relugolix characterized by a PXRD pattern substantially as illustrated in FIG. 9.

Fourteenth aspect of the present application provides a process for preparing crystalline form R2 of relugolix comprising:

a) providing a mixture of relugolix in a mixture of water and a suitable organic solvent;
b) stirring the mixture for sufficient time; and
c) isolating crystalline form R2 of relugolix.

Fifteenth aspect of the present application relates to a composition comprising crystalline form R2 of relugolix and one or more pharmaceutically acceptable excipient.

Sixteenth aspect of the present application relates to crystalline form R3 of relugolix characterized by its powder X-ray diffraction (PXRD) pattern having peaks at 9.423 and 19.342±0.2° 2θ. In embodiments, the present application provides crystalline form R3 of relugolix characterized by its PXRD pattern having additional peaks located at 12.933, 15.853, 20.723, 24.963±0.2° 2θ.

Seventeenth aspect of the present application provides crystalline form R3 of relugolix characterized by a PXRD pattern substantially as illustrated in FIG. 10.

Eighteenth aspect of the present application provides a process for preparing crystalline form R3 of relugolix comprising:

a) providing a mixture of relugolix in an aromatic hydrocarbon solvent;
b) stirring the mixture for sufficient time;
c) isolating crystalline form R3 of relugolix.

Nineteenth aspect of the present application relates to a composition comprising crystalline form R3 of relugolix and one or more pharmaceutically acceptable excipient.

Twentieth of the present application relates to crystalline form R4 of relugolix characterized by its powder X-ray diffraction (PXRD) pattern having peaks at 12.794 and 19.612±0.2° 2θ. In embodiments, the present application provides crystalline form R4 of relugolix characterized by its PXRD pattern having additional peaks located at 8.405, 9.923, 10.479, 11.470 and 25.477±0.2° 2θ.

Twenty first aspect of the present application provides crystalline form R4 of relugolix characterized by a PXRD pattern substantially as illustrated in FIG. 11.

Twenty second aspect of the present application provides a process for preparing crystalline form R4 of relugolix comprising:

a) providing a mixture of relugolix in an aromatic hydrocarbon solvent;
b) stirring the mixture for sufficient time;
c) isolating crystalline form R4 of relugolix.

Twenty third of the present application relates to a composition comprising crystalline form R4 of relugolix and one or more pharmaceutically acceptable excipient.

Twenty fourth aspect of the present application relates to crystalline form R5 of relugolix characterized by its powder X-ray diffraction (PXRD) pattern having peaks at 7.671 and 19.268±0.2° 2θ. In embodiments, the present application provides crystalline form R5 of relugolix characterized by its PXRD pattern having additional peaks located at 6.186, 16.753, 17.451, 21.852 and 25.072±0.2° 2θ.

Twenty fifth of the present application provides crystalline form R5 of relugolix characterized by a PXRD pattern substantially as illustrated in FIG. 12.

Twenty sixth aspect of the present application provides a process for preparing crystalline form R5 of relugolix comprising:

a) providing a mixture of relugolix in o-xylene;
b) stirring the mixture for sufficient time; and
c) isolating crystalline form R5 of relugolix.

Twenty seventh aspect of the present application relates to a composition comprising crystalline form R5 of relugolix and one or more pharmaceutically acceptable excipient.

Twenty eighth aspect of the present application relates to crystalline form R6 of relugolix characterized by its powder X-ray diffraction (PXRD) pattern having peaks at 12.624 and 19.515±0.2° 2θ. In embodiments, the present application provides crystalline form R6 of relugolix characterized by its PXRD pattern having additional peaks located at 8.319, 9.825, 11.365, 16.904, 17.526 and 25.313±0.2° 2θ.

Twenty ninth aspect of the present application provides crystalline form R6 of relugolix characterized by a PXRD pattern substantially as illustrated in FIG. 13.

Thirtieth aspect of the present application provides a process for preparing crystalline form R6 of relugolix comprising:

a) providing a mixture of relugolix in p-xylene;
b) stirring the mixture for sufficient time; and
c) isolating crystalline form R6 of relugolix.

Thirty first aspect of the present application relates to a composition comprising crystalline form R6 of relugolix and one or more pharmaceutically acceptable excipient.

Thirty second aspect of the present application relates to crystalline form R7 of relugolix characterized by its powder X-ray diffraction (PXRD) pattern having peaks at 5.329, 12.596, 19.282 and 20.593±0.2° 2θ. In embodiments, the present application provides crystalline form R7 of relugolix characterized by its PXRD pattern having additional peaks located at 6.286, 10.517, 15.441, 15.978, 24.194, 25.976 and 28.678±0.2° 2θ.

Thirty third aspect of the present application provides crystalline form R7 of relugolix characterized by a PXRD pattern substantially as illustrated in FIG. 14.

Thirty fourth aspect of the present application provides a process for preparing crystalline form R7 of relugolix comprising:

a) providing a mixture of relugolix in a mixture of water and alcohol;
b) stirring the mixture for sufficient time; and
c) isolating crystalline form R7 of relugolix.

Thirty fifth aspect of the present application relates to a composition comprising crystalline form R7 of relugolix and one or more pharmaceutically acceptable excipient.

Thirty sixth aspect of the present application relates to crystalline form R8 of relugolix characterized by its powder X-ray diffraction (PXRD) pattern having peaks at 6.50 and 7.68±0.2° 2θ. In embodiments, the present application provides crystalline form R8 of relugolix characterized by its PXRD pattern having additional peaks located at 15.42, 19.02 and 23.28±0.2° 2θ.

Thirty seventh aspect of the present application provides crystalline form R8 of relugolix characterized by a PXRD pattern substantially as illustrated in FIG. 15.

Thirty eighth aspect of the present application provides a process for preparing crystalline form R8 of relugolix comprising:

a) providing a solution of relugolix in benzyl alcohol;
b) adding a suitable anti-solvent to the solution of step a); and
c) isolating crystalline form R8 of relugolix.

Thirty ninth aspect of the present application relates to a composition comprising crystalline form R8 of relugolix and one or more pharmaceutically acceptable excipient.

Fortieth aspect of the present application relates to crystalline form R9 of relugolix characterized by its powder X-ray diffraction (PXRD) pattern having peaks at 6.51, 8.01, 18.34 and 19.47±0.2° 2θ.

Forty first aspect of the present application provides crystalline form R9 of relugolix characterized by a PXRD pattern substantially as illustrated in FIG. 16.

Forty second aspect of the present application provides a process for preparing crystalline form R9 of relugolix comprising:

a) providing a mixture of relugolix in a ketone solvent;
b) mixing the solution of step a) with an ether solvent;
c) isolating crystalline form R9 of relugolix.

Forty third aspect of the present application relates to a composition comprising crystalline form R9 of relugolix and one or more pharmaceutically acceptable excipient.

Forty fourth aspect of the present application relates to crystalline form R10 of relugolix characterized by its powder X-ray diffraction (PXRD) pattern having peaks at 6.55, 8.06 and 19.58±0.2° 2θ. In embodiments, the present application provides crystalline form R10 of relugolix characterized by its PXRD pattern having additional peaks located at 12.74, 13.03 and 24.63±0.2° 2θ.

Forty fifth aspect of the present application provides crystalline form R10 of relugolix characterized by a PXRD pattern substantially as illustrated in FIG. 17.

Forty sixth aspect of the present application provides a process for preparing crystalline form R10 of relugolix comprising drying crystalline form R9 under suitable condition.

Forty seventh aspect of the present application provides a process for preparing crystalline form R10 of relugolix comprising:

a) dissolving relugolix in a suitable solvent;
b) adding an ether solvent to the step a);
c) isolating crystalline form R10 of relugolix.

Forty eighth of the present application relates to a composition comprising crystalline form R10 of relugolix and one or more pharmaceutically acceptable excipient.

### BRIEF DESCRIPTION OF THE DRAWING

FIG. 1: The PXRD pattern of amorphous form of relugolix obtained by the process of example 1

FIG. 2: The PXRD pattern of amorphous solid dispersion of relugolix and HPMC-AS (Grade LG) obtained by the process of example 2

FIG. 3: The PXRD pattern of amorphous solid dispersion of relugolix and co-povidone obtained by the process of example 3

FIG. 4: The PXRD pattern of amorphous solid dispersion of relugolix and HPC obtained by the process of example 4

FIG. 5: The PXRD pattern of amorphous solid dispersion of relugolix and povidone K-30 obtained by the process of example 5

FIG. 6: The PXRD pattern of amorphous solid dispersion of relugolix and Eudragit obtained by the process of example 6

FIG. 7: The PXRD pattern of amorphous solid dispersion of relugolix and HPMC-AS (Grade MG) obtained by the process of example 7

FIG. 8: The PXRD pattern of crystalline form R1 of relugolix

FIG. 9: The PXRD pattern of crystalline form R2 of relugolix

FIG. 10: The PXRD pattern of crystalline form R3 of relugolix

FIG. 11: The PXRD pattern of crystalline form R4 of relugolix

FIG. 12: The PXRD pattern of crystalline form R5 of relugolix

FIG. 13: The PXRD pattern of crystalline form R6 of relugolix

FIG. 14: The PXRD pattern of crystalline form R7 of relugolix

FIG. 15: The PXRD pattern of crystalline form R8 of relugolix

FIG. 16: The PXRD pattern of crystalline form R9 of relugolix

FIG. 17: The PXRD pattern of crystalline form R10 of relugolix

FIG. 18: The TGA pattern of crystalline form R1 of relugolix

FIG. 19: The DSC pattern of crystalline form R1 of relugolix

FIG. 20: The TGA pattern of crystalline form R8 of relugolix

FIG. 21: The DSC pattern of crystalline form R8 of relugolix

### DETAILED DESCRIPTION

First aspect of the present application relates to amorphous form of relugolix.

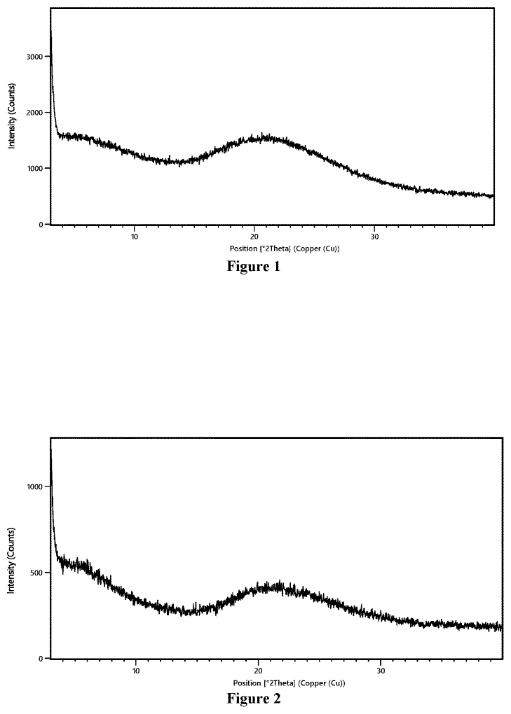

One of the embodiments of the present application relates to amorphous form of relugolix characterized by a PXRD pattern substantially as illustrated in the pattern of FIG. 1.

Second aspect of the present application relates to process for preparing amorphous form of relugolix comprising
(a) dissolving relugolix in a suitable solvent or mixture thereof,
(b) removing the solvent of step (a) by any suitable technique, and
(c) optionally drying amorphous relugolix.

Any crystalline form of relugolix or mixture thereof may be used as starting material for preparing amorphous form of relugolix.

In embodiments of step (a), suitable solvents include, but are not limited to ketone solvent such as acetone, ethyl methyl ketone, 2-butanone, methyl isobutyl ketone and the like; ether solvent such as tetrahydrofuran, dioxane and the like; ester solvent such as ethyl acetate, isopropyl acetate and the like; aromatic hydrocarbon solvent such as toluene, xylene and the like; halogenated hydrocarbon solvent such as dichloromethane, chloroform and the like; alcohol solvent such as methanol, ethanol, propanol, isopropanol and the like; and mixtures thereof. Specifically, the solvent may be selected from a group of halogenated hydrocarbon solvent such as dichloromethane, chloroform and the like. Specifically, the solvent may be dichloromethane.

In one embodiment of step (a), relugolix may be dissolved in a suitable solvent at a temperature of about 5° C. to about boiling point of the solvent. In a specific embodiment, relugolix may be dissolved in a suitable solvent at a temperature of about 20° C. to about 30° C.

In another embodiment of step (a), the solution of relugolix may be filtered to remove any undissolved particles or extraneous matter.

In one embodiment of step (b), suitable techniques that may be used for the removal of solvent include but are not limited to rotational distillation using a device such as Buchi Rotavapor, spray drying, agitated thin film drying (“ATFD”), freeze drying (lyophilization), Rotary cone vacuum dryer (RVPD), melt crystallization and the like, optionally under reduced pressure. One specific embodiment of the present application relates to spray-drying or freeze-drying technique, to provide amorphous form of relugolix. Another specific embodiment of the present application relates to using Buchi Rotavapor to provide amorphous form of relugolix. Alternatively, an anti-solvent may be added to the solution of relugolix of step (a) to precipitate amorphous form of relugolix and the precipitated solid may be isolated by any methods known in the art, such as filtration. The suitable anti-solvent may be any organic solvent known in the art in which relugolix is insoluble or slightly soluble. The suitable anti-solvent may also be water.

The resulting solid may be collected by using techniques such as by scraping, or by shaking the container, or other techniques specific to the equipment used.

The isolated solid may optionally be further dried to afford amorphous form of relugolix. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, specifically at temperatures less than about 80° C. and more specifically less than about 60° C. The drying may be carried out for any time period required for obtaining a desired product quality, such as from about 30 minutes to about 24 hours, or longer.

The dried product may optionally be subjected to a particle size reduction procedure to produce desired particle sizes and distributions. Milling or micronization may be performed before drying, or after the completion of drying of the product. Equipment that may be used for particle size reduction includes but not limited to ball mill, roller mill, hammer mill, and jet mill.

The amorphous form of relugolix that is substantially free of any crystalline form is hereby referred to as ‘pure amorphous’ form of relugolix. Pure amorphous form of relugolix does not contain more than about 10% of any crystalline form of relugolix. Specifically, pure amorphous form of relugolix does not contain more than about 5% of any crystalline form of relugolix. More specifically, pure amorphous form of relugolix does not contain more than about 3% of any crystalline form of relugolix. Most specifically, pure amorphous form of relugolix does not contain more than about 1% of any crystalline form of relugolix. FIG. 1 illustrates XRPD pattern of pure amorphous form of relugolix obtained by a process of example 1.

It was found that the amorphous relugolix is stable and has excellent physico-chemical properties. The amorphous form of relugolix of the present application may be easily formulated into a pharmaceutical composition along with suitable pharmaceutically acceptable excipients.

Third aspect of the present application relates to a pharmaceutical composition comprising amorphous form of relugolix and one or more pharmaceutically acceptable excipient. Pharmaceutical composition comprising amorphous form of relugolix of the present application may be formulated as: solid oral dosage forms such as, but not limited to, powders, granules, pellets, tablets, and capsules; liquid oral dosage forms such as, but not limited to, syrups, suspensions, dispersions, and emulsions; and injectable preparations such as, but not limited to, solutions, dispersions, and freeze dried compositions. Pharmaceutical composition may be in the forms of immediate release, delayed release, or modified release. Further, immediate release compositions may be conventional, dispersible, chewable, mouth dissolving, or flash melt preparations; and modified release compositions that may comprise hydrophilic or hydrophobic, or combinations of hydrophilic and hydrophobic release rate controlling substances to form matrix or reservoir or combination of matrix and reservoir systems. The compositions may be prepared using any one or more of techniques such as direct blending, dry granulation, wet granulation, extrusion and spheronization. Compositions may be presented as uncoated, film coated, sugar coated, powder coated, enteric coated, and modified release coated.

Fourth aspect of the present application relates to amorphous solid dispersion of relugolix with one or more pharmaceutically acceptable carrier.

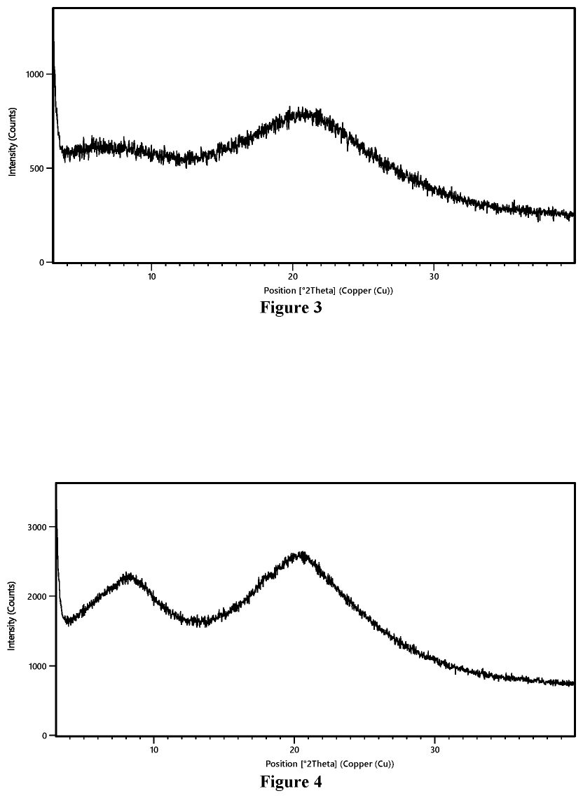

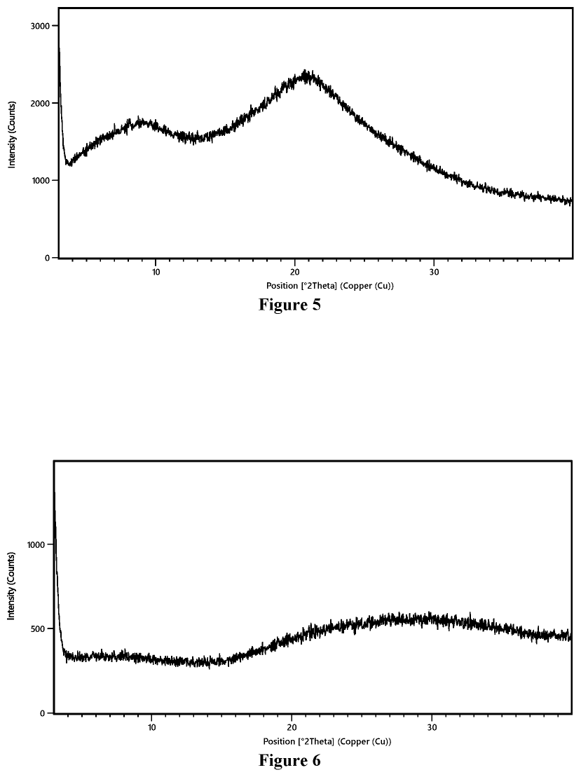

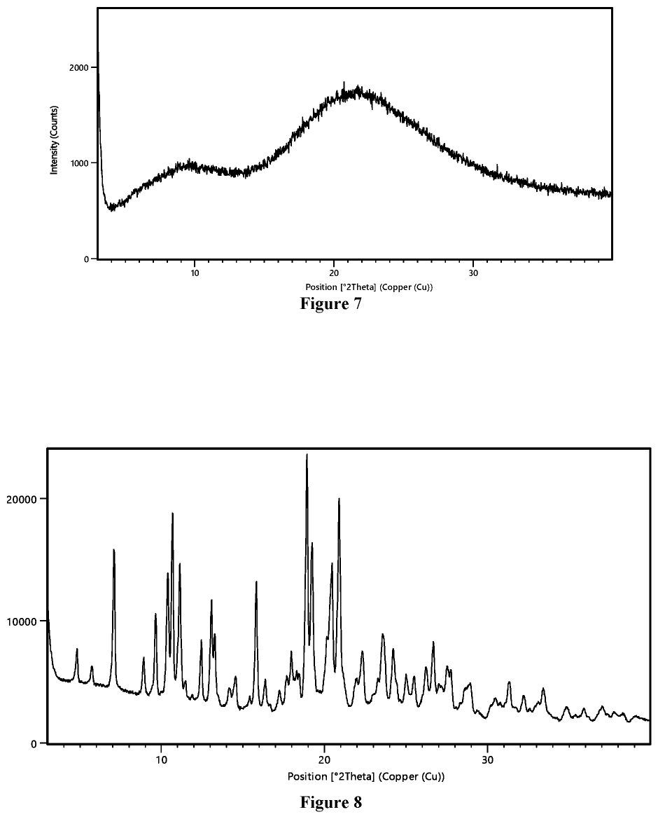

Another aspect of the present application relates to amorphous form of relugolix characterized by a PXRD pattern substantially as illustrated in the pattern of FIG. 2 or FIG. 3 or FIG. 4 or FIG. 5 or FIG. 6 or FIG. 7.

The pharmaceutically acceptable carrier may be any suitable carrier reported in the literature. Specifically, the pharmaceutically acceptable carrier includes, but not restricted to methyl cellulose, ethyl cellulose, hydroxypropyl methylcellulose, hydroxypropyl methylcellulose acetate succinate, hydroxypropyl cellulose, polysaccharides, heteropolysaccharides (pectins), poloxamers, poloxamines, ethylene vinyl acetates, polyethylene glycols, dextrans, polyvinyl alcohols, propylene glycols, polyvinylacetates, phosphatidylcholines (lecithins), miglyols, polylactic acid, polyhydroxybutyric acid, polyvinylpyrrolidones (PVP), silicon dioxide (syloid), copovidone, methacrylic acid, polymethacrylate, mixtures of two or more thereof, copolymers thereof and derivatives thereof. More specifically, the pharmaceutically acceptable carrier may be selected from a group of hydroxypropyl methylcellulose (HPMC), hydroxypropyl methylcellulose acetate succinate (HPMC-AS), hydroxypropyl cellulose (HPC), co-povidone, polyvinylpyrrolidones (PVP), Eudragit, and mixtures thereof.

The ratio (weight/weight) of relugolix and pharmaceutically acceptable carrier in amorphous solid dispersion of the present application may be about 5:95, or about 10:90, or about 15:85, or about 20:80, or about 25:75, or about 30:70, or about 35:65, or about 40:60, or about 45:55, or about 50:50 and vice versa.

Fifth aspect of the present application relates to process for preparation of amorphous solid dispersion of relugolix with one or more pharmaceutically acceptable carrier comprising
(a) dissolving relugolix and one or more pharmaceutically acceptable carrier in suitable solvent or mixture thereof;
(b) isolating amorphous solid dispersion of relugolix;
(c) optionally drying the amorphous solid dispersion of relugolix.

Any crystalline form of relugolix or mixture thereof may be used as starting material for preparing amorphous form of relugolix.

In embodiments of step (a), suitable solvents include, but are not limited to ketone solvent such as acetone, ethyl methyl ketone, 2-butanone, methyl isobutyl ketone and the like; ether solvent such as tetrahydrofuran, dioxane and the like; ester solvent such as ethyl acetate, isopropyl acetate and the like; aromatic hydrocarbon solvent such as toluene, xylene and the like; halogenated hydrocarbon solvent such as dichloromethane, chloroform and the like; alcohol solvent such as methanol, ethanol, propanol, isopropanol and the like; mixtures thereof. In one embodiment, the solvent may be a mixture of a ketone solvent and a halogenated hydrocarbon solvent. In another embodiment, the solvent may be a mixture of acetone and dichloromethane. In yet another embodiment, the solvent may be a mixture of an alcohol solvent, a ketone solvent and a halogenated hydrocarbon solvent. In still another embodiment, the solvent may be a mixture of methanol, acetone and dichloromethane.

In one embodiment of step (a), relugolix may be dissolved in a suitable solvent at a temperature of about 0° C. to about boiling point of the solvent. In another embodiment of step (a), relugolix may be dissolved at about 30° C. to about 50° C.

In another embodiment of step (a), the solution of relugolix may be filtered to remove any undissolved particles or extraneous matter.

Isolation of amorphous solid dispersion of relugolix may involve one or more methods including removal of solvent by techniques known in the art e.g. evaporation, distillation, filtration of precipitated solid and the like, cooling, concentrating the reaction mass, and the like. Stirring or other alternate methods such as shaking, agitation, and the like, may also be employed for the isolation. One of the embodiments relates to addition of an anti-solvent to the solution of step (a) to precipitate amorphous solid dispersion of relugolix with one or more pharmaceutically acceptable carrier. Another embodiment relates to distillation of solvent at atmospheric pressure or above, or under reduced pressures and at a temperatures less than about 120° C., less than about 100° C., less than about 90° C., or any other suitable temperatures. Any temperature and vacuum conditions can be used as long as there is no increase in the impurity levels of the product due to decomposition.

Suitable techniques which can be used for the distillation include, but not limited to, distillation using a rotary evaporator device such as a Buchi Rotavapor, spray drying, agitated thin film drying (“ATFD”), and the like. Specifically, techniques providing a rapid solvent removal may be utilized to provide the desired amorphous solid dispersion of relugolix with one or more pharmaceutically acceptable carrier. More specifically, distillation using a rota-vapor device such as a Buchi Rotavapor or a spray drying technique may be used for the isolation of amorphous solid dispersion of relugolix with one or more pharmaceutically acceptable carrier.

The solid may be collected using techniques such as by scraping, or by shaking the container, or other techniques specific to the equipment used.

The isolated solid may optionally be further dried to afford amorphous solid dispersion of relugolix. Drying may be suitably carried out using any of an air tray dryer, vacuum tray dryer, fluidized bed dryer, spin flash dryer, flash dryer and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained amorphous solid dispersion of relugolix with one or more pharmaceutically acceptable carrier may optionally be subjected to a particle size reduction procedure to produce desired particle sizes and distributions. Milling or micronization may be performed before drying, or after the completion of drying of the amorphous solid dispersions. Equipment that may be used for particle size reduction include, but not limited to, ball, roller, and hammer mills, jet mills and the like.

It was found that the amorphous solid dispersion of relugolix of the present application is stable and has excellent physico-chemical properties. The amorphous solid dispersion of relugolix of the present application may be easily formulated into a pharmaceutical composition comprising relugolix along with one or more pharmaceutically acceptable excipients.

Sixth aspect of the present application relates to a pharmaceutical composition comprising amorphous solid dispersion of relugolix and one or more pharmaceutically acceptable excipient.

Pharmaceutical composition comprising amorphous solid dispersion of relugolix of the present application may be formulated as: solid oral dosage forms such as, but not limited to, powders, granules, pellets, tablets, and capsules; liquid oral dosage forms such as, but not limited to, syrups, suspensions, dispersions, and emulsions; and injectable preparations such as, but not limited to, solutions, dispersions, and freeze dried compositions. Pharmaceutical composition may be in the forms of immediate release, delayed release, or modified release. Further, immediate release compositions may be conventional, dispersible, chewable, mouth dissolving, or flash melt preparations; and modified release compositions that may comprise hydrophilic or hydrophobic, or combinations of hydrophilic and hydrophobic release rate controlling substances to form matrix or reservoir or combination of matrix and reservoir systems. The compositions may be prepared using any one or more of techniques such as direct blending, dry granulation, wet granulation, extrusion and spheronization. Compositions may be presented as uncoated, film coated, sugar coated, powder coated, enteric coated, and modified release coated.

Seventh aspect of the present application relates to crystalline form R1 of relugolix characterized by its powder X-ray diffraction (PXRD) pattern having peaks at 7.076, 9.632, 13.048, 15.795 and 20.872±0.2° 2θ. In embodiments, the present application provides crystalline form R1 of relugolix characterized by its PXRD pattern having additional peaks located at 10.665, 12.424, 18.892, 19.216, 23.544, 26.612 and 26.641±0.2° 2θ.

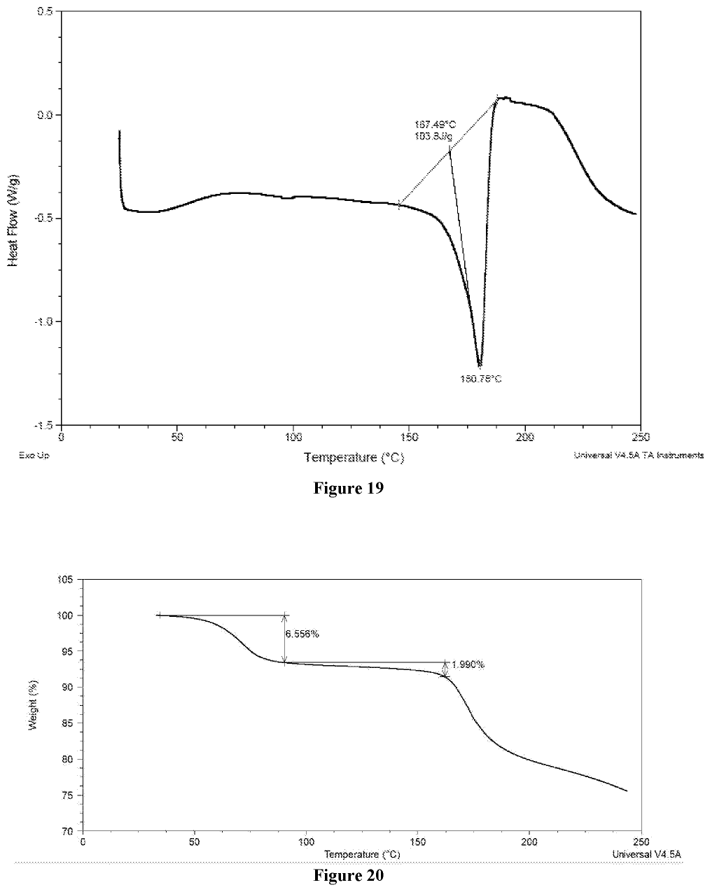

Eighth aspect of the present application provides crystalline form R1 of relugolix characterized by a PXRD pattern substantially as illustrated in FIG. 8. One embodiment of the present application provides crystalline form R1 of relugolix characterized by a TGA pattern substantially as illustrated in FIG. 18. Another embodiment of the present application provides crystalline form R1 of relugolix characterized by a DSC pattern substantially as illustrated in FIG. 19. Crystalline form R1 of relugolix may be an anhydrate.

Ninth aspect of the present application provides a process for preparing crystalline form R1 of relugolix comprising:

a) providing a solution of relugolix in a suitable solvent or mixtures thereof;
b) stirring the solution for sufficient time; and
c) isolating crystalline form R1 of relugolix.
The suitable solvent of step a), includes but not limited to, nitrile solvent such as acetonitrile, propionitrile and the like; halogenated hydrocarbon solvent such as dichloromethane, chloroform and the like; ketone solvent such as acetone, methyl isobutyl ketone and the like; or mixture thereof. In one of the embodiments, the solvent of step a) may be a nitrile solvent. More specifically, the solvent of step a) may be acetonitrile. In another embodiment, the solvent of step a) may be a ketone solvent. Specifically, the solvent of step a) may be acetone. In yet another embodiment of step a), any physical form of relugolix may be utilized, which may be crystalline or amorphous. In yet another embodiment of step a), any physical form of relugolix may be utilize. In still another embodiment of step a), the mixture of relugolix and the solvent may be heated from about 40° C. to about boiling point of the solvent to provide a solution of relugolix in a suitable solvent or mixtures thereof.

In embodiments of step b), the solution of step a) may be stirred at about 0° C. to about boiling point of the solvent for about 30 minutes to 10 hours. Specifically, the solution of step a) may be stirred at about room temperature for about 1 hour to 3 hours. In another embodiment of step b), the solution of step a) may be filtered to remove any undissolved material. In yet another embodiment of step b), an anti-solvent may be added to the solution of step b). The anti-solvent may be such in which relugolix has no solubility or minimal solubility.

In one embodiment of step b), the seed crystals of crystalline form R1 of relugolix may be optionally added to the solution of step a) or step b).

Isolation of crystalline form R1 of relugolix from the mixture of step b) may be performed by any technique known in the art. Specifically, crystalline form R1 of relugolix may be isolated from the mixture of step b) by filtration. Optionally, the crystalline form R1 of relugolix may be dried under suitable condition. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R1 of relugolix may optionally be subjected to a particle size reduction procedure to produce desired particle sizes and distributions. Milling or micronization may be performed before drying, or after the completion of drying of crystalline form R1 of relugolix. Equipment that may be used for particle size reduction includes but not limited to ball mill, roller mill, hammer mill, and jet mill.

The crystalline form R1 of relugolix of the present application is stable and has excellent physico-chemical properties. The crystalline form R1 of relugolix is stable for at least three months at 40° C. and 75% RH (relative humidity), at 25° C. and 60% RH (relative humidity) and at 2-8° C. The crystalline form R1 of relugolix of the present application may be easily formulated into a pharmaceutical composition comprising relugolix.

Tenth aspect of the present application relates to a composition comprising crystalline form R1 of relugolix and one or more pharmaceutically acceptable excipient.

Eleventh aspect of the present application relates to a process for purification of relugolix comprising the steps of:

a) providing a mixture of crude relugolix and a suitable solvent;
b) optionally adding an anti-solvent to the mixture of step a);
c) isolating pure relugolix.

In embodiments of step a), the suitable solvent may be selected from a group of an ether solvent and a nitrile solvent. The ether solvent includes but not limited to diethyl ether, tetrahydrofuran, methyl tert-butyl ether, mixtures thereof and the like. The nitrile solvent includes but not limited to acetonitrile, propionitrile and the like. In one specific embodiment, the solvent is tetrahydrofuran. In another specific embodiment, the solvent is acetonitrile. In yet another embodiment of step a), the mixture of relugolix and the suitable solvent may be provided in a temperature of about 0° C. to about boiling point of the solvent. Specifically, the mixture may be heated to a temperature of about 40° C. to about boiling point of the solvent.

In embodiments of step b), a suitable anti-solvent may be added optionally to the mixture of step a). The anti-solvent may include but not limited to water. Water may be added optionally to the mixture of step a) at a temperature of about 0° C. to about boiling point of the solvent.

In embodiments of step c), the mixture of step b) may be stirred at a temperature of about 0° C. to about boiling point of the solvent for a sufficient time before isolating pure relugolix. Specifically, the mixture of step b) may be stirred at a temperature of about 0° C. to about 40° C. for about 1 hour to 30 hours before isolating pure relugolix.

Isolation of pure relugolix in step c) may be performed by any technique known in the art. Specifically, pure relugolix may be isolated from the mixture of step b) by filtration. Optionally, the pure relugolix may be dried under suitable condition. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, specifically at temperatures less than about 80° C. and more specifically less than about 60° C. and most specifically at 40° C. The drying may be carried out for any time period required for obtaining a desired product quality, such as from about 5 minutes to about 24 hours, or longer.

Twelfth aspect of the present application relates to crystalline form R2 of relugolix characterized by its powder X-ray diffraction (PXRD) pattern having peaks at 9.404, 18.032 and 19.059±0.2° 2θ. In embodiments, the present application provides crystalline form R2 of relugolix characterized by its PXRD pattern having additional peaks located at 7.438, 10.133, 12.058, 16.488, 21.022 and 23.062±0.2° 2θ.

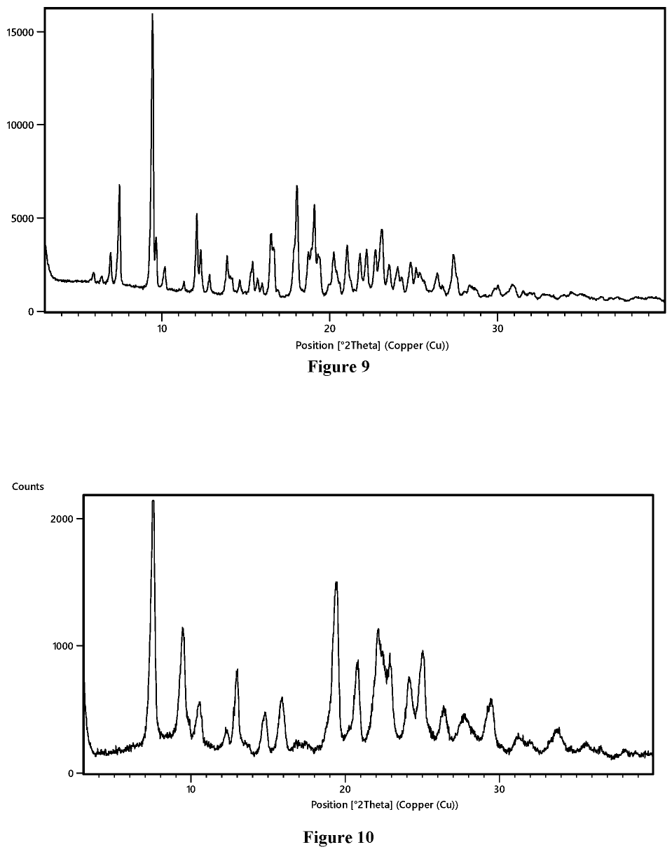

Thirteenth aspect of the present application provides crystalline form R2 of relugolix characterized by a PXRD pattern substantially as illustrated in FIG. 9.

The crystalline form R2 of relugolix may be a hydrate.

Fourteenth aspect of the present application provides a process for preparing crystalline form R2 of relugolix comprising:

a) providing a mixture of relugolix in a mixture of water and a suitable organic solvent;
b) stirring the mixture for sufficient time; and
c) isolating crystalline form R2 of relugolix.

The suitable organic solvent of step a), includes but not limited to, nitrile solvent such as acetonitrile, propionitrile and the like; alcohol solvent such as methanol, ethanol, isopropanol and the like; ketone solvent such as acetone, methyl isobutyl ketone and the like; ether solvent such as tetrahydrofuran, dioxane and the like. Specifically, the suitable organic solvent of step a) may be selected from a group of alcohol solvent and ketone solvent. In one embodiment, the suitable organic solvent of step a) may be acetone. In another embodiment, the suitable organic solvent of step a) may be alcohol solvent selected from a group of methanol, ethanol and isopropanol. In yet another embodiment of step a), any physical form of relugolix may be utilized, which may be crystalline or amorphous. In embodiment of step b), the solution of step a) may be filtered to remove any undissolved material. In another embodiments of step b), the mixture of step a) may be stirred at about 0° C. to about boiling point of the solvent for about 30 minutes to 10 hours. Specifically, the mixture of step a) may be stirred at about room temperature for about 1 hour to 6 hours.

In one embodiment of step b), the seed crystals of crystalline form R2 of relugolix may be optionally added to the mixture of step a) or step b).

Isolation of crystalline form R2 of relugolix from the mixture of step b) may be performed by any technique known in the art. Specifically, crystalline form R2 of relugolix may be isolated from the mixture of step b) by filtration. Optionally, the crystalline form R2 of relugolix may be dried under suitable condition. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R2 of relugolix may optionally be subjected to a particle size reduction procedure to produce desired particle sizes and distributions. Milling or micronization may be performed before drying, or after the completion of drying of crystalline form R2 of relugolix. Equipment that may be used for particle size reduction includes but not limited to ball mill, roller mill, hammer mill, and jet mill.

The crystalline form R2 of relugolix of the present application is stable and has excellent physico-chemical properties. The crystalline form R2 of relugolix of the present application may be easily formulated into a pharmaceutical composition comprising relugolix.

Still another aspect of the present application relates to a composition comprising crystalline form R2 of relugolix and one or more pharmaceutically acceptable excipient.

Sixteenth aspect of the present application relates to crystalline form R3 of relugolix characterized by its powder X-ray diffraction (PXRD) pattern having peaks at 9.423 and 19.342±0.2° 2θ. In embodiments, the present application provides crystalline form R3 of relugolix characterized by its PXRD pattern having additional peaks located at 12.933, 15.853, 20.723, 24.963±0.2° 2θ.

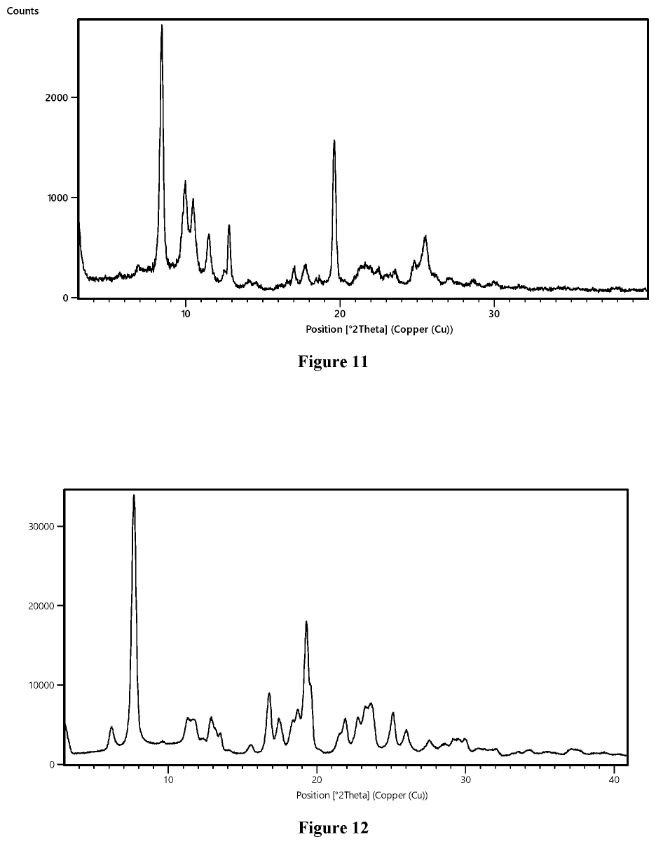

Seventeenth aspect of the present application provides crystalline form R3 of relugolix characterized by a PXRD pattern substantially as illustrated in FIG. 10.

Eighteenth aspect of the present application provides a process for preparing crystalline form R3 of relugolix comprising:

a) providing a mixture of relugolix in an aromatic hydrocarbon solvent;
b) stirring the mixture for sufficient time;
c) isolating crystalline form R3 of relugolix.

The suitable organic solvent of step a), includes but not limited to, benzene, xylene, toluene and the like. Specifically, the suitable organic solvent of step a) may be xylene. In yet another embodiment of step a), any physical form of relugolix may be utilized, which may be crystalline or amorphous. Specifically, crystalline form of relugolix may be utilized. More specifically, crystalline form R1 may be utilized. In embodiment of step b), the mixture of step a) may be stirred at about 0° C. to about boiling point of the solvent for about 30 minutes to 10 hours. Specifically, the mixture of step a) may be stirred at about room temperature for about 1 hour to 6 hours.

In one embodiment of step b), the seed crystals of crystalline form R3 of relugolix may be optionally added to the mixture of step a) or step b).

Isolation of crystalline form R3 of relugolix from the mixture of step b) may be performed by any technique known in the art. Specifically, crystalline form R3 of relugolix may be isolated from the mixture of step b) by filtration. Crystalline form R3 of relugolix may be dried under suitable condition for about 30 minutes to about 4 hours. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R3 of relugolix may optionally be subjected to a particle size reduction procedure to produce desired particle sizes and distributions. Milling or micronization may be performed before drying, or after the completion of drying of crystalline form R3 of relugolix. Equipment that may be used for particle size reduction includes but not limited to ball mill, roller mill, hammer mill, and jet mill.

The crystalline form R3 of relugolix of the present application is stable and has excellent physico-chemical properties. The crystalline form R3 of relugolix of the present application may be easily formulated into a pharmaceutical composition comprising relugolix.

Nineteenth aspect of the present application relates to a composition comprising crystalline form R3 of relugolix and one or more pharmaceutically acceptable excipient.

Twentieth aspect of the present application relates to crystalline form R4 of relugolix characterized by its powder X-ray diffraction (PXRD) pattern having peaks at 12.794 and 19.612±0.2° 2θ. In embodiments, the present application provides crystalline form R4 of relugolix characterized by its PXRD pattern having additional peaks located at 8.405, 9.923, 10.479, 11.470 and 25.477±0.2° 2θ.

Twenty first aspect of the present application provides crystalline form R4 of relugolix characterized by a PXRD pattern substantially as illustrated in FIG. 11.

Twenty second aspect of the present application provides a process for preparing crystalline form R4 of relugolix comprising:

a) providing a mixture of relugolix in an aromatic hydrocarbon solvent;
b) stirring the mixture for sufficient time;
c) isolating crystalline form R4 of relugolix.
The suitable organic solvent of step a), includes but not limited to, benzene, xylene, toluene and the like. Specifically, the suitable organic solvent of step a) may be xylene. In yet another embodiment of step a), any physical form of relugolix may be utilized, which may be crystalline or amorphous. Specifically, crystalline form of relugolix may be utilized. More specifically, crystalline form R1 may be utilized. In embodiment of step b), the mixture of step a) may be stirred at about 0° C. to about boiling point of the solvent for about 30 minutes to 10 hours. Specifically, the mixture of step a) may be stirred at about room temperature for about 1 hour to 6 hours.

In one embodiment of step b), the seed crystals of crystalline form R4 of relugolix may be optionally added to the mixture of step a) or step b).

Isolation of crystalline form R4 of relugolix from the mixture of step b) may be performed by any technique known in the art. Specifically, crystalline form R4 of relugolix may be isolated from the mixture of step b) by filtration. Crystalline form R4 of relugolix may be dried under suitable condition for about 12 hours to about 60 hours. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R4 of relugolix may optionally be subjected to a particle size reduction procedure to produce desired particle sizes and distributions. Milling or micronization may be performed before drying, or after the completion of drying of crystalline form R4 of relugolix. Equipment that may be used for particle size reduction includes but not limited to ball mill, roller mill, hammer mill, and jet mill.

The crystalline form R4 of relugolix of the present application is stable and has excellent physico-chemical properties. The crystalline form R4 of relugolix of the present application may be easily formulated into a pharmaceutical composition comprising relugolix.

Twenty third of the present application relates to a composition comprising crystalline form R4 of relugolix and one or more pharmaceutically acceptable excipient.

Twenty fourth aspect of the present application relates to crystalline form R5 of relugolix characterized by its powder X-ray diffraction (PXRD) pattern having peaks at 7.671 and 19.268±0.2° 2θ. In embodiments, the present application provides crystalline form R5 of relugolix characterized by its PXRD pattern having additional peaks located at 6.186, 16.753, 17.451, 21.852 and 25.072±0.2° 2θ.

Twenty fifth aspect of the present application provides crystalline form R5 of relugolix characterized by a PXRD pattern substantially as illustrated in FIG. 12.

Twenty sixth aspect of the present application provides a process for preparing crystalline form R5 of relugolix comprising:

a) providing a mixture of relugolix in o-xylene;
b) stirring the mixture for sufficient time; and
c) isolating crystalline form R5 of relugolix.

In one embodiment of step a), any physical form of relugolix may be utilized, which may be crystalline or amorphous. In yet another embodiment of step a), any physical form of relugolix may be utilize. Specifically, crystalline form R1 of relugolix may be utilized.

In embodiments of step b), the mixture of step a) may be stirred at about 0° C. to about boiling point of the solvent for about 30 minutes to 10 hours. Specifically, the solution of step a) may be stirred at about room temperature for about 2 hours to 5 hours.

In one embodiment of step b), the seed crystals of crystalline form R5 of relugolix may be optionally added to the solution of step a) or step b).

Isolation of crystalline form R5 of relugolix from the mixture of step b) may be performed by any technique known in the art. Specifically, crystalline form R5 of relugolix may be isolated from the mixture of step b) by filtration. Optionally, the crystalline form R5 of relugolix may be dried under suitable condition. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R5 of relugolix may optionally be subjected to a particle size reduction procedure to produce desired particle sizes and distributions. Milling or micronization may be performed before drying, or after the completion of drying of crystalline form R5 of relugolix. Equipment that may be used for particle size reduction includes but not limited to ball mill, roller mill, hammer mill, and jet mill.

The crystalline form R5 of relugolix of the present application is stable and has excellent physico-chemical properties. The crystalline form R5 of relugolix of the present application may be easily formulated into a pharmaceutical composition comprising relugolix.

Twenty seventh aspect of the present application relates to a composition comprising crystalline form R5 of relugolix and one or more pharmaceutically acceptable excipient.

Twenty eighth aspect of the present application relates to crystalline form R6 of relugolix characterized by its powder X-ray diffraction (PXRD) pattern having peaks at 12.624 and 19.515±0.2° 2θ. In embodiments, the present application provides crystalline form R6 of relugolix characterized by its PXRD pattern having additional peaks located at 8.319, 9.825, 11.365, 16.904, 17.526 and 25.313±0.2° 2θ.

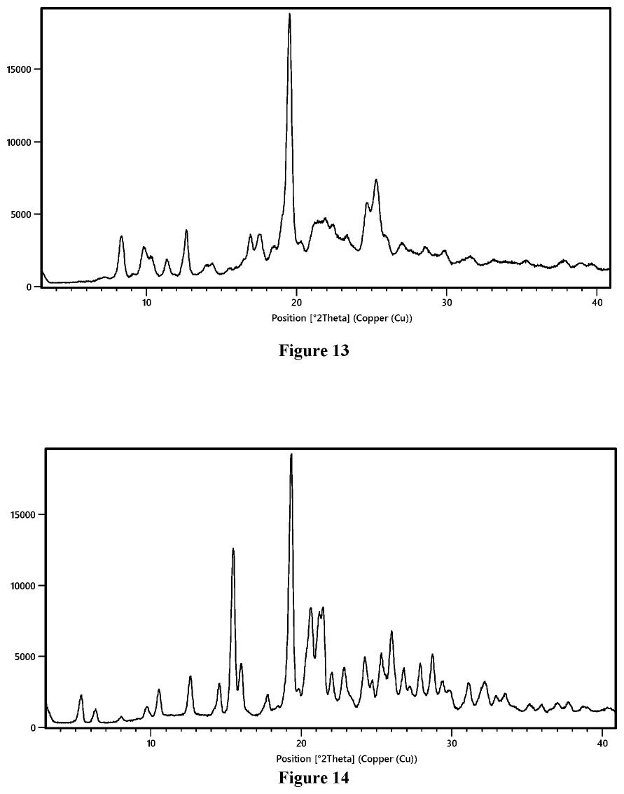

Twenty ninth aspect of the present application provides crystalline form R6 of relugolix characterized by a PXRD pattern substantially as illustrated in FIG. 13.

Thirtieth aspect of the present application provides a process for preparing crystalline form R6 of relugolix comprising:

a) providing a mixture of relugolix in p-xylene;
b) stirring the mixture for sufficient time; and
c) isolating crystalline form R6 of relugolix.

In one embodiment of step a), any physical form of relugolix may be utilized, which may be crystalline or amorphous. In yet another embodiment of step a), any physical form of relugolix may be utilize. Specifically, crystalline form R1 of relugolix may be utilized.

In embodiments of step b), the mixture of step a) may be stirred at about 0° C. to about boiling point of the solvent for about 30 minutes to 10 hours. Specifically, the solution of step a) may be stirred at about room temperature for about 2 hours to 5 hours.

In one embodiment of step b), the seed crystals of crystalline form R6 of relugolix may be optionally added to the mixture of step a) or step b).

Isolation of crystalline form R6 of relugolix from the mixture of step b) may be performed by any technique known in the art. Specifically, crystalline form R6 of relugolix may be isolated from the mixture of step b) by filtration. Optionally, the crystalline form R6 of relugolix may be dried under suitable condition. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R6 of relugolix may optionally be subjected to a particle size reduction procedure to produce desired particle sizes and distributions. Milling or micronization may be performed before drying, or after the completion of drying of crystalline form R6 of relugolix. Equipment that may be used for particle size reduction includes but not limited to ball mill, roller mill, hammer mill, and jet mill.

The crystalline form R6 of relugolix of the present application is stable and has excellent physico-chemical properties. The crystalline form R6 of relugolix of the present application may be easily formulated into a pharmaceutical composition comprising relugolix.

Thirty first aspect of the present application relates to a composition comprising crystalline form R6 of relugolix and one or more pharmaceutically acceptable excipient.

Thirty second aspect of the present application relates to crystalline form R7 of relugolix characterized by its powder X-ray diffraction (PXRD) pattern having peaks at 5.329, 12.596, 19.282 and 20.593±0.2° 2θ. In embodiments, the present application provides crystalline form R7 of relugolix characterized by its PXRD pattern having additional peaks located at 6.286, 10.517, 15.441, 15.978, 24.194, 25.976 and 28.678±0.2° 2θ.

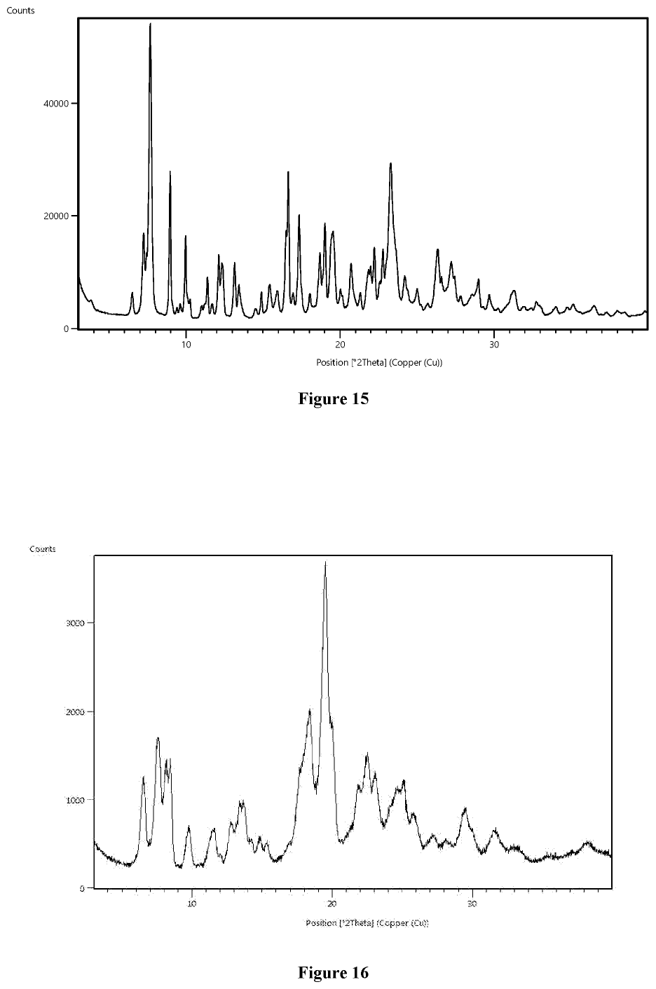

Thirty third aspect of the present application provides crystalline form R7 of relugolix characterized by a PXRD pattern substantially as illustrated in FIG. 14.

Thirty fourth aspect of the present application provides a process for preparing crystalline form R7 of relugolix comprising:

a) providing a mixture of relugolix in a mixture of water and alcohol;
b) stirring the mixture for sufficient time; and
c) isolating crystalline form R7 of relugolix.

In one embodiment of step a), any physical form of relugolix may be utilized, which may be crystalline or amorphous. In another embodiment of step a), the alcohol solvent may include but not limited to methanol, ethanol, isopropanol, n-butanol and mixture thereof. Specifically, the alcohol solvent may be methanol.

In embodiments of step b), the mixture of step a) may be stirred at about 0° C. to about boiling point of the solvent for about 30 minutes to 10 hours. Specifically, the solution of step a) may be stirred at about room temperature for about 2 hours to 5 hours.

In one embodiment of step b), the seed crystals of crystalline form R7 of relugolix may be optionally added to the mixture of step a) or step b).

Isolation of crystalline form R7 of relugolix from the mixture of step b) may be performed by any technique known in the art. Specifically, crystalline form R7 of relugolix may be isolated from the mixture of step b) by filtration. Optionally, the crystalline form R6 of relugolix may be dried under suitable condition. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R7 of relugolix may optionally be subjected to a particle size reduction procedure to produce desired particle sizes and distributions. Milling or micronization may be performed before drying, or after the completion of drying of crystalline form R7 of relugolix. Equipment that may be used for particle size reduction includes but not limited to ball mill, roller mill, hammer mill, and jet mill.

The crystalline form R7 of relugolix of the present application is stable and has excellent physico-chemical properties. The crystalline form R7 of relugolix of the present application may be easily formulated into a pharmaceutical composition comprising relugolix.

Thirty fifth aspect of the present application relates to a composition comprising crystalline form R7 of relugolix and one or more pharmaceutically acceptable excipient.

Thirty sixth aspect of the present application relates to crystalline form R8 of relugolix characterized by its powder X-ray diffraction (PXRD) pattern having peaks at 6.50 and 7.68±0.2° 2θ. In embodiments, the present application provides crystalline form R8 of relugolix characterized by its PXRD pattern having additional peaks located at 15.42, 19.02 and 23.28±0.2° 2θ.

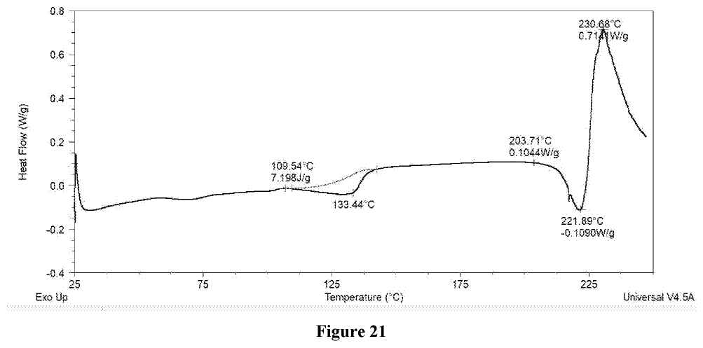

Thirty seventh aspect of the present application provides crystalline form R8 of relugolix characterized by a PXRD pattern substantially as illustrated in FIG. 15. One embodiment of the present application provides crystalline form R8 of relugolix characterized by a TGA pattern substantially as illustrated in FIG. 20. Another embodiment of the present application provides crystalline form R8 of relugolix characterized by a DSC pattern substantially as illustrated in FIG. 21. Crystalline form R8 of relugolix may be hemi-benzyl alcohol solvate The crystalline form R8 of relugolix may contain about 4.0% to about 10.0% (w/w) of benzyl alcohol content, as measured by GC.

Thirty eighth aspect of the present application provides a process for preparing crystalline form R8 of relugolix comprising:

a) providing a solution of relugolix in benzyl alcohol;
b) adding a suitable anti-solvent to the solution of step a); and
c) isolating crystalline form R8 of relugolix.

In one embodiment of step a), a mixture of relugolix and benzyl alcohol may be stirred for a sufficient time until a clear solution is obtained. In another embodiment of step a), a mixture of relugolix and benzyl alcohol may be heated to obtain a clear solution. Specifically, a mixture of relugolix and benzyl alcohol may be heated up to 50° C. for sufficient time to provide a solution of step a). In yet another embodiment of step a), any physical form of relugolix may be utilized, which may be crystalline or amorphous. In another embodiment of step b), the solution of step a) may be filtered to remove any undissolved material.

In embodiments of step b), the anti-solvent may be selected from a group of an ether solvent, a hydrocarbon solvent and mixture thereof. The ether solvent may include but not limited to diethyl ether, diisopropyl ether, methyl tert-butyl ether and like. The hydrocarbon solvent may include but not limited to n-heptane, n-hexane and the like. In one embodiment, the anti-solvent may be methyl tert-butyl ether. In another embodiment, the anti-solvent may be a mixture of methyl tert-butyl ether and n-heptane. In one embodiment, the anti-solvent may be added to the solution of step a). In yet another embodiment, the solution of step a) may be added to the anti-solvent.

Isolation of crystalline form R8 of relugolix from the mixture of step b) may be performed by any technique known in the art. Specifically, crystalline form R8 of relugolix may be isolated from the mixture of step b) by filtration. Optionally, the crystalline form R8 of relugolix may be dried under suitable condition. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R8 of relugolix may optionally be subjected to a particle size reduction procedure to produce desired particle sizes and distributions. Milling or micronization may be performed before drying, or after the completion of drying of crystalline form R1 of relugolix. Equipment that may be used for particle size reduction includes but not limited to ball mill, roller mill, hammer mill, and jet mill.

The crystalline form R8 of relugolix of the present application is stable and has excellent physico-chemical properties. The crystalline form R8 of relugolix of the present application may be easily formulated into a pharmaceutical composition comprising relugolix.

Thirty ninth aspect of the present application relates to a composition comprising crystalline form R8 of relugolix and one or more pharmaceutically acceptable excipient.

Fortieth aspect of the present application relates to crystalline form R9 of relugolix characterized by its powder X-ray diffraction (PXRD) pattern having peaks at 6.51, 8.01, 18.34 and 19.47±0.2° 2θ.

Forty first aspect of the present application provides crystalline form R9 of relugolix characterized by a PXRD pattern substantially as illustrated in FIG. 16.

Forty second aspect of the present application provides a process for preparing crystalline form R9 of relugolix comprising:

a) providing a mixture of relugolix in a ketone solvent;
b) mixing the solution of step a) with an ether solvent;
c) isolating crystalline form R9 of relugolix.

The suitable ketone solvent of step a), includes but not limited to, acetone, methyl ethyl ketone, cyclopentanone and the like. Specifically, the suitable ketone solvent of step a) may be methyl ethyl ketone. In another embodiment of step a), any physical form of relugolix may be utilized, which may be crystalline or amorphous. Specifically, crystalline form of relugolix may be utilized. More specifically, crystalline form R1 may be utilized. In yet another embodiment of step a), the mixture of ketone solvent and relugolix may be heated up to reflux temperature to provide a solution of relugolix in a ketone solvent.

In one embodiment of step b), the ether solvent includes not limited to, diethyl ether, tetrahydrofuran, methyl tert-butyl ether and the like. Specifically, the suitable ether solvent of step a) may be methyl tert-butyl ether. In another embodiment of step b), the solution of relugolix in ketone solvent may be added to the ether solvent. Alternatively, the ether solvent may be added to the solution of relugolix in ketone solvent. In yet another embodiment of step b), the seed crystals of crystalline form R9 of relugolix may be optionally added to the mixture of step a) or step b).

Isolation of crystalline form R9 of relugolix from the mixture of step b) may be performed by any technique known in the art. Specifically, crystalline form R9 of relugolix may be isolated from the mixture of step b) by filtration. Crystalline form R9 of relugolix may be dried under suitable condition for about 30 minutes to about 4 hours. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R9 of relugolix may optionally be subjected to a particle size reduction procedure to produce desired particle sizes and distributions. Milling or micronization may be performed before drying, or after the completion of drying of crystalline form R9 of relugolix. Equipment that may be used for particle size reduction includes but not limited to ball mill, roller mill, hammer mill, and jet mill.

The crystalline form R9 of relugolix of the present application is stable and has excellent physico-chemical properties. The crystalline form R9 of relugolix of the present application may be easily formulated into a pharmaceutical composition comprising relugolix.

Forty third aspect of the present application relates to a composition comprising crystalline form R9 of relugolix and one or more pharmaceutically acceptable excipient.

Forty fourth aspect of the present application relates to crystalline form R10 of relugolix characterized by its powder X-ray diffraction (PXRD) pattern having peaks at 6.55, 8.06 and 19.58±0.2° 2θ. In embodiments, the present application provides crystalline form R10 of relugolix characterized by its PXRD pattern having additional peaks located at 12.74, 13.03 and 24.63±0.2° 2θ.

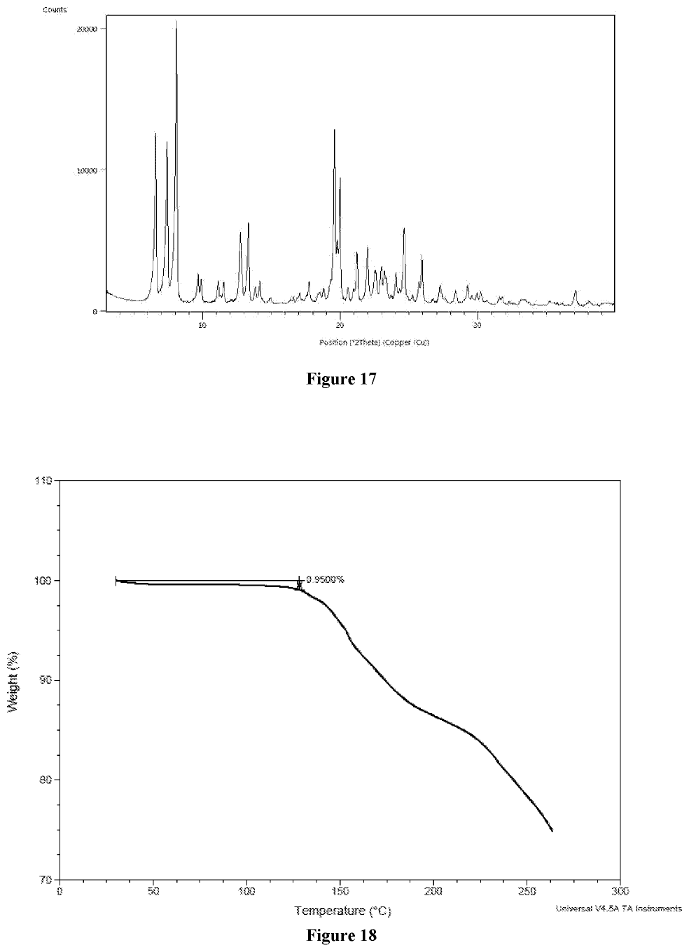

Forty fifth aspect of the present application provides crystalline form R10 of relugolix characterized by a PXRD pattern substantially as illustrated in FIG. 17. Crystalline form R10 of relugolix may be an anhydrate.

Forty sixth aspect of the present application provides a process for preparing crystalline form R10 of relugolix comprising drying crystalline form R9 under suitable condition.

Crystalline form R9 of relugolix may be dried under suitable condition for about 1 hour to about 10 hours. Drying may be suitably carried out using any of an air tray dryer, vacuum tray dryer, fluidized bed dryer, spin flash dryer, flash dryer, and the like. The drying may be carried out at atmospheric pressure or above, or under reduced pressure, specifically at temperatures more than about 100° C. and more specifically more than about 120° C. and most specifically at 130° C.

Forty seventh aspect of the present application provides a process for preparing crystalline form R10 of relugolix comprising:

a) dissolving relugolix in a suitable solvent;
b) adding an ether solvent to the step a);
c) isolating crystalline form R10 of relugolix.

The suitable solvent of step a), includes but not limited to, ketone solvent such as acetone, methyl ethyl ketone, cyclopentanone and the like; a nitrile solvent such as acetonitrile, propionitrile, and the like; an ester solvent such as ethyl acetate, methyl acetate and the like. Specifically, the suitable solvent of step a) is a ketone solvent. More specifically, the ketone solvent of step a) may be methyl ethyl ketone. In another embodiment of step a), any physical form of relugolix may be utilized, which may be crystalline or amorphous. Specifically, crystalline form of relugolix may be utilized. More specifically, crystalline form R1 may be utilized. In yet another embodiment of step a), the mixture of ketone solvent and relugolix may be heated up to reflux temperature to provide a solution of relugolix in a ketone solvent.

In one embodiment of step b), the ether solvent includes not limited to, diethyl ether, tetrahydrofuran, methyl tert-butyl ether and the like. Specifically, the suitable ether solvent of step a) may be methyl tert-butyl ether. In another embodiment of step b), the solution of relugolix in ketone solvent may be added to the ether solvent. Alternatively, the ether solvent may be added to the solution of relugolix in ketone solvent.

In yet another embodiment, the seed crystals of crystalline form R10 of relugolix may be optionally added to the mixture of step a) or step b).

Isolation of crystalline form R10 of relugolix from the mixture of step b) may be performed by any technique known in the art. Specifically, crystalline form R10 of relugolix may be isolated from the mixture of step b) by filtration. Crystalline form R10 of relugolix may be dried under suitable condition for about 30 minutes to about 10 hours. Drying may be suitably carried out using any of an air tray dryer, vacuum tray dryer, fluidized bed dryer, spin flash dryer, flash dryer, and the like. The drying may be carried out at atmospheric pressure or above, or under reduced pressures, at temperatures less than about 120° C., less than about 100° C., less than about 80° C., or any other suitable temperatures. The drying may be carried out for any time period required for obtaining a desired product quality, such as from about 30 minutes to about 24 hours, or longer.

The obtained crystalline form R10 of relugolix may optionally be subjected to a particle size reduction procedure to produce desired particle sizes and distributions. Equipment that may be used for particle size reduction includes but not limited to ball mill, roller mill, hammer mill, and jet mill.

The crystalline form R10 of relugolix of the present application is stable and has excellent physico-chemical properties. The crystalline form R10 of relugolix of the present application may be easily formulated into a pharmaceutical composition comprising relugolix.

Forty eighth aspect of the present application relates to a composition comprising crystalline form R10 of relugolix and one or more pharmaceutically acceptable excipient.

One aspect of the present application provides pharmaceutically acceptable dosage form comprising crystalline form R1 of relugolix and one or more pharmaceutically acceptable excipients. Crystalline form R1 or R2 or R3 or R4 or R5 or R6 or R7 or R8 or R9 or R10 of relugolix together with one or more pharmaceutically acceptable excipients of the present application may be formulated as: solid oral dosage forms such as, but not limited to, powders, granules, pellets, tablets, and capsules; liquid oral dosage forms such as, but not limited to, syrups, suspensions, dispersions, and emulsions; and injectable preparations such as, but not limited to, solutions, dispersions, and freeze dried compositions. Formulations may be in the forms of immediate release, delayed release, or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using any one or more of techniques such as direct blending, dry granulation, wet granulation, and extrusion and spheronization. Compositions may be presented as uncoated, film coated, sugar coated, powder coated, enteric coated, and modified release coated.

Pharmaceutically acceptable excipients that are useful in the present application include, but are not limited to: diluents such as starches, pregelatinized starches, lactose, powdered celluloses, microcrystalline celluloses, dicalcium phosphate, tricalcium phosphate, mannitol, sorbitol, sugar, and the like; binders such as acacia, guar gum, tragacanth, gelatin, polyvinylpyrrolidones, hydroxypropyl celluloses, hydroxypropyl methyl celluloses, pregelatinized starches, and the like; disintegrants such as starches, sodium starch glycolate, pregelatinized starches, crospovidones, croscarmellose sodium, colloidal silicon dioxide, and the like; lubricants such as stearic acid, magnesium stearate, zinc stearate, and the like; glidants such as colloidal silicon dioxide and the like; solubility or wetting enhancers such as anionic, cationic, or neutral surfactants; complex forming agents such as various grades of cyclodextrins and resins; and release rate controlling agents such as hydroxypropyl celluloses, hydroxymethyl celluloses, hydroxypropyl methylcelluloses, ethylcelluloses, methylcelluloses, various grades of methyl methacrylates, waxes, and the like. Other pharmaceutically acceptable excipients that are useful include, but are not limited to, film formers, plasticizers, colorants, flavoring agents, sweeteners, viscosity enhancers, preservatives, antioxidants, and the like.

The PXRD conditions for the measurement of PXRD peaks of relugolix of the present application are as follows:

Range: 3° 2θ to 40° 2θ in conventional reflection mode

### Instrument: PANalytical X-ray Diffractometer

### Detector: X'celerator

Source: Copper K-alpha radiation (1.5418 Angstrom).

### Definitions

The following definitions are used in connection with the present application unless the context indicates otherwise.

The terms “about,” “general, ‘generally,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

The term “anhydrous” or “anhydrate” in relation to crystalline forms of relugolix, relates to a crystalline form of relugolix, which does not include water and any other solvent in a defined, stoichiometric amount within the crystal. Moreover, an “anhydrous” or “anhydrate” form would typically not contain more than 1% (w/w) of water or organic solvents, as measured by TGA.

The term “solvate” in relation to crystalline forms of relugolix, relates to a crystalline form of relugolix, which incorporates a solvent in crystalline structure, either in a stoichiometric or in a non-stoichiometric manner.

All percentages and ratios used herein are by weight of the total composition and all measurements made are at about 25° C. and about atmospheric pressure, unless otherwise designated. All temperatures are in degrees Celsius unless specified otherwise. As used herein, the terms “comprising” and “comprises” mean the elements recited, or their equivalents in structure or function, plus any other element or elements which are not recited. The terms “having” and “including” are also to be construed as open ended. All ranges recited herein include the endpoints, including those that recite a range between two values. Whether so indicated or not, all values recited herein are approximate as defined by the circumstances, including the degree of expected experimental error, technique error, and instrument error for a given technique used to measure a value.

The term “optional” or “optionally” is taken to mean that the event or circumstance described in the specification may or may not occur, and that the description includes instances where the event occurs and instances where it does not.

Certain specific aspects and embodiments of the present application will be explained in greater detail with reference to the following examples, which are provided only for purposes of illustration and should not be construed as limiting the scope of the disclosure in any manner.

### EXAMPLES

### Example 1: Preparation of Amorphous Form of Relugolix

Relugolix (500 mg) was dissolved in dichloromethane (20 mL) at 25° C. The solvent was evaporated using Rotavapour under vacuum at 150 rpm and 50° C. to provide the desired compound.

### Example 2: Preparation of Amorphous Solid Dispersion of Relugolix with HPMC-AS (Grade: LG)

Relugolix (500 mg) and HPMC-AS (Grade: LG) (250 mg) was dissolved in a mixture of acetone (30 mL) and dichloromethane (20 mL) at 40° C. The solution was filtered to make it particle-free. The solvent was evaporated under vacuum at 175 rpm and at 50° C. for 15 minutes. The isolated solid was dried to provide the desired compound.

### Example 3: Preparation of Amorphous Solid Dispersion of Relugolix with Co-Povidone

Relugolix (500 mg) and co-povidone (Grade: VA-64) was dissolved in a mixture of acetone (40 mL) and dichloromethane (10 mL) at 40° C. The solution was filtered to make it particle-free. The filtrate was evaporated vacuum at 175 rpm and at 50° C. The solid was dried at 50° C. for 15 minutes to provide the title compound.

### Example 4: Preparation of Amorphous Solid Dispersion of Relugolix with HPC

Relugolix (500 mg) and HPC (Grade: LG) (250 mg) was dissolved in a mixture of acetone (40 mL) and dichloromethane (20 mL) at 40° C. The solution was filtered to make it particle-free. The solvent was evaporated under vacuum at 175 rpm and at 50° C. The solid was dried at 50° C. for 115 minutes to provide the desired compound.

### Example 5: Preparation of Amorphous Solid Dispersion of Relugolix with Povidone K-30

Relugolix (500 mg) and povidone (Grade: K-30) (250 mg) was dissolved in a mixture of acetone (40 mL) and dichloromethane (20 mL) at 40° C. The solution was filtered to make it particle-free. The solvent was evaporated under vacuum at 175 rpm and at 50° C. The solid was dried at 50° C. for 15 minutes to provide the title compound.

### Example 6: Preparation of Amorphous Solid Dispersion of Relugolix with Eudragit

Relugolix (500 mg) and Eudragit (Grade: L-100) (250 mg) was mixed in a mixture of acetone (30 mL) and dichloromethane (20 mL) at 40° C. A clear solution was obtained by addition of methanol (20 mL) at the same temperature. The solution was filtered to make it particle-free. The solvent was evaporated under vacuum at 175 rpm and at 50° C. The solid was dried at 50° C. for 20 minutes to provide the title compound.

### Example 7: Preparation of Amorphous Solid Dispersion of Relugolix with HPMC-AS (Grade: MG)

Relugolix (500 mg) and HPMC-AS (Grade: MG) (250 mg) was dissolved in a mixture of acetone (40 mL) and dichloromethane (20 mL) at 40° C. The solution was filtered to make it particle-free. The filtrate was evaporated in a rotavapor at 175 rpm and at 50° C. for 15 minutes to provide the desired compound.

### Example 8: Preparation of Crystalline Form R1 of Relugolix

Crude relugolix (35 g) having a purity of 98.20% by HPLC was dissolved in acetonitrile (700 mL) and the undissolved solid was filtered out. The reaction mass was stirred at room temperature for 1 hour and the precipitated solid was filtered out. The solid was washed with acetonitrile (35 mL) and dried in a hot air oven at 40° C. for 3 hours to get the desired compound. The PXRD pattern of the desired compound matches with crystalline form R1, as provided in FIG. 8.

### Yield: 28.5 g

### Purity (By HPLC): 99.29%

### Example 9: Preparation of Crystalline Form R1 of Relugolix

Crude relugolix (45 g) was taken in acetonitrile (1500 mL) and stirred at room temperature for 1 hour. The solid was filtered out, dried and re-dissolved in dichloromethane (1125 mL). The solution was washed with water (3×375 mL) and concentrated under vacuum at 45° C. The solid obtained was suspended in acetonitrile (975 mL), stirred for 1 h at room temperature and filtered. The solid was washed with acetonitrile and dried in a hot air oven at 40° C. for 3 hours to provide the desired compound. The PXRD pattern of the desired compound matches with crystalline form R1, as provided in FIG. 8.

### Yield: 34 g

### Purity (By HPLC): 99.15%

### Example 10: Preparation of Pure Relugolix

A mixture of crude relugolix (1.5 g) having a purity of 98.32% by HPLC and tetrahydrofuran (7.5 mL) was provided. The mixture was heated to reflux and stirred for 30 minutes at the reflux temperature. Water (1 mL) was added to the above mixture at the reflux temperature and stirred for 30 minutes. A clear solution appeared. The reaction mass was cooled to room temperature and stirred for 24 hours. The mixture was then further cooled to 5° C. and stirred for 30 minutes. The solid was filtered, washed with tetrahydrofuran (1.5 mL) and dried at 50° C. for 45 minutes to afford the title compound.

### Yield: 0.55 g

### Purity (By HPLC): 99.59%

### Example 11: Preparation of Pure Relugolix

A mixture of crude relugolix (10 g) having a purity of 97.5% by HPLC and tetrahydrofuran (50 mL) was provided. The mixture was heated to reflux, stirred for 1 h at the reflux temperature, cooled to 5° C. and stirred at the same temperature for 2 h. The mixture was thereafter again heated to reflux and water (20 mL) was added. The reaction mass was cooled to room temperature and further amount of water (30 mL) was added. The reaction mass was further cooled to 5° C. and stirred for 1 hour. The solid was filtered and dried under vacuum.
The solid obtained above (3.0 g) was taken in tetrahydrofuran (12 mL). The reaction mass was heated to reflux and stirred for 20 minutes at the reflux temperature. Water (12 mL) was added to the above mixture at the reflux temperature. The reaction mass was cooled to room temperature and stirred for 2 hours. The solid was filtered and dried at room temperature under vacuum to afford the title compound.

### Yield: 1.50 g

### Purity (By HPLC): 98.86%

### Example 12: Preparation of Crystalline Form R2 of Relugolix

A mixture of amorphous relugolix (200 mg), water (2 mL) and methanol (1 mL) was stirred at 31° C. for 4 hours. The solid was filtered and dried at room temperature for 1 hour to provide the desired compound.

### Example 13: Preparation of Crystalline Form R2 of Relugolix

A mixture of amorphous relugolix (200 mg), water (2 mL) and acetone (1 mL) was stirred at 31° C. for 4 hours. The solid was filtered and dried at room temperature for 1 hour to provide the desired compound.

### Example 14: Preparation of Crystalline Form R2 of Relugolix

A mixture of amorphous relugolix (200 mg), water (2 mL) and isopropanol (1 mL) was stirred at 31° C. for 4 hours. The solid was filtered and dried at room temperature for 1 hour to provide the desired compound.

### Example 15: Preparation of Crystalline Form R3 of Relugolix

A mixture of crystalline form R1 of relugolix (300 mg) and xylene (1 mL) was stirred at 25-27° C. for 4 hours. The solid was filtered and dried at 30° C. for 1 hour to provide the desired compound.

### Example 16: Preparation of Crystalline Form R4 of Relugolix

A mixture of crystalline form R1 of relugolix (300 mg) and xylene (1 mL) was stirred at 25-27° C. for 4 hours. The solid was filtered and dried at 30° C. for 2 days to provide the desired compound.

### Example 17: Preparation of Crystalline Form R5 of Relugolix

Crystalline form R1 relugolix (0.5 g) was mixed with o-xylene (6 mL). The mixture was stirred at about 25-27° C. for 3.5 hours. The solid was filtered and dried at room temperature for 2 hours to afford the title compound.

### Example 18: Preparation of Crystalline Form R6 of Relugolix

Crystalline form R1 relugolix (0.5 g) was mixed with p-xylene (6 mL). The mixture was stirred at about 25-27° C. for 3.5 hours. The solid was filtered and dried at room temperature for 2 hours to afford the title compound.

### Example 19: Preparation of Crystalline Form R7 of Relugolix

Relugolix (0.5 g) was mixed with a mixture of water (6 mL) and methanol (3 mL). The mixture was stirred at about 25-28° C. for 3 hours. The solid was filtered and dried at 40° C. for 4 hours to afford the title compound.

### Example 20: Preparation of Crystalline Form R8 of Relugolix

A mixture of relugolix (1.05 g) and benzyl alcohol (3 mL) was heated to 50° C. to provide a clear solution. The solution was cooled to 0° C. and methyl tert-butyl ether (30 mL) was added slowly and the reaction mass was stirred for about 3 hours at 0° C. The precipitated solid was filtered and dried in a vacuum tray drier at room temperature to provide the desired compound.
Benzyl alcohol content: 5.97% (w/w)

### Example 21: Preparation of Crystalline Form R8 of Relugolix

A mixture of relugolix (1.03 g) and benzyl alcohol (3 mL) was heated to 50° C. to provide a clear solution. The solution was cooled to 0° C. and a mixture of methyl tert-butyl ether (20 mL) and n-heptane (20 mL) was added slowly. The reaction mass was stirred for about 3 hours at 0° C. The precipitated solid was filtered to provide the desired compound.
Benzyl alcohol content: 7.37% (w/w)

### Example 22: Preparation of Crystalline Form R9 of Relugolix

A mixture of crystalline form R1 of relugolix (2 g) and methyl ethyl ketone (20 mL) was heated at 65° C. and stirred for about 1.5 hours. The solution was cooled to 25° C. and filtered. The solution was added to pre-cooled methyl tert-butyl ether (60 mL) at −5° C. and the resulting mixture was stirred for 2 hours at −5° C. and then at 25° C. for 1 hour. The solid was filtered and dried at 25° C. for 4 hours under vacuum to provide the title compound.

### Example 23: Preparation of Crystalline Form R10 of Relugolix

Crystalline form R9 of relugolix was dried at 130° C. for 2 hours to provide the desired compound.

### Example 24: Preparation of Crystalline Form R1 of Relugolix

A mixture of relugolix (2.5 g) and acetone (40 mL) was heated to 50° C. for 1 hour and filtered to make the solution particle free. The solution was slowly cooled to 40° C. and 50 mg of crystalline form R1 of relugolix was added to the solution. The solution was further cooled to 25° C. and kept at that temperature for 2.5 hours. The precipitated crystalline form R1 of relugolix was filtered to provide the title compound.

### Example 25: Preparation of Crystalline Form R10 of Relugolix

A mixture of relugolix (2 g) and methyl ethyl ketone (10 mL) was heated at 55° C. to prepare a solution. The solution was filtered to make it particle free. The solution was cooled to −5° C. and the above solution was added to pre-cooled methyl tert-butyl ether (50 mL). The mixture was stirred for 2 hours and slowly warmed to room temperature (25° C.). The precipitated compound was filtered and dried in a vacuum tray drier at 75° C. for 1 hour to afford the title compound.

### Example 26: Preparation of Crystalline Form R10 of Relugolix

A mixture of relugolix (2 g) and methyl ethyl ketone (20 mL) was heated at 65° C. for 30 minutes to prepare a solution. The solution was filtered to make it particle free. The solution was cooled to −5° C. and the above solution was added to pre-cooled methyl tert-butyl ether (60 mL). The mixture was stirred for 5 hours. The precipitated compound was filtered to afford the title compound.

We claim:

1. A crystalline form R1 of relugolix characterized by powder X-ray diffraction (PXRD) pattern having peaks at 7.076, 9.632, 13.048, 15.795 and 20.872±0.2° 2θ.

2. The crystalline form R1 of relugolix, as claimed in claim 1, characterized by powder X-ray diffraction (PXRD) pattern having additional peaks at 10.665, 12.424, 18.892, 19.216, 23.544, 26.612 and 26.641±0.2° 2θ.

3. The crystalline form R1 of relugolix, as claimed in claim 1, characterized by a PXRD pattern substantially as illustrated in FIG. 8.

4. A process for preparation of crystalline form R1 of relugolix, as claimed in claim 1, comprising:
a) providing a solution of relugolix in a suitable solvent or mixtures thereof;
b) stirring the solution for sufficient time; and
c) isolating crystalline form R1 of relugolix.

5. The process of claim 4, wherein the solvent is a nitrile solvent.

6. The process of claim 5, wherein the nitrile solvent is acetonitrile.

7. The process of claim 4, wherein the solvent acetone.

8. A crystalline form R10 of relugolix characterized by powder X-ray diffraction (PXRD) pattern having peaks at 6.55, 8.06 and 19.58±0.2° 2θ.

9. The crystalline form R10 of relugolix, as claimed in claim 7, characterized by powder X-ray diffraction (PXRD) pattern having additional peaks at 12.74, 13.03 and 24.63±0.2° 2θ.

10. The crystalline form R10 of relugolix, as claimed in claim 8, characterized by a PXRD pattern substantially as illustrated in FIG. 17.

11. A process for preparation of crystalline form R10 of relugolix, as claimed in claim 8, comprising
a) dissolving relugolix in a suitable solvent;
b) adding an ether solvent to the step a);
c) isolating crystalline form R10 of relugolix.

12. The process of claim 11, wherein the suitable solvent in step a) is a ketone solvent.

13. The process of claim 12, wherein the ketone solvent is methyl ethyl ketone.

14. The process of claim 11, wherein the ether solvent in step b) is methyl tert-butyl ether.

15. A process for preparation of crystalline form R10 of relugolix, as claimed in claim 8, comprising drying crystalline form R9 under suitable condition.
